(19) United States
(12) Patent Application Publication
(10) Pub. No.: US 2026/0077170 A1
(43) Pub. Date: Mar. 19, 2026

(54) MICRONEEDLE DRUG DELIVERY APPARATUS AND PREPARATION METHOD THEREOF

(71) Applicant: TSINGHUA UNIVERSITY, Beijing (CN)

(72) Inventors: Mingjun ZHANG, Beijing (CN); Xize GAO, Beijing (CN); Jiacong LI, Beijing (CN); Jing LI, Beijing (CN); Jing XU, Beijing (CN); Mingxiao XIE, Beijing (CN); Hongru LIU, Beijing (CN); Longxi REN, Beijing (CN)

(21) Appl. No.: 19/403,032

(22) Filed: Nov. 27, 2025

Related U.S. Application Data
(63) Continuation of application No. PCT/CN2023/097550, filed on May 31, 2023.

(51) Int. Cl. A61M 37/00

(52) U.S. Cl. CPC A61M 37/0015; A61M 2037/0023; A61M 2037/0046; A61M 2037/0053; A61M 2037/0061; A61M 2205/02; A61M 2210/106

(57) ABSTRACT

The present application relates to the technical field of drug delivery, and particularly, to a microneedle drug delivery apparatus and a preparation method thereof. The apparatus includes: a substrate; an array of microneedles loaded on the substrate; and a liquid-absorbing swellable material wrapped by the substrate to form a closed sandwich structure. This microneedle drug delivery apparatus allows microneedles to penetrate an intestinal wall using a mild water-absorbing swellable mechanism and natural peristaltic behavior of a gastrointestinal tract, to complete drug delivery. It penetrates a physiological barrier of oral delivery and absorption of biologic drugs by physical means, improving oral bioavailability of biologic drugs. Also, all materials used are biodegradable soft materials, with high safety.

### CROSS-REFERENCE TO RELATED APPLICATIONS

This application is a continuation of International Application No. PCT/CN2023/097550, filed on May 31, 2023, which is incorporated herein by reference in its entirety.

### FIELD

The present disclosure relates to the technical field of drug delivery, and particularly, to a microneedle drug delivery apparatus and a preparation method thereof.

### BACKGROUND

Oral administration, due to its advantages such as simple administration and high patient compliance, has always been a popular drug delivery method. However, such an approach is not suitable for biologic macromolecular drugs such as peptides, proteins, nucleic acids, antibodies, etc. Currently, injection administration, including intramuscular injection, intravenous injection, etc., is still the main delivery method for the biologic macromolecular drugs. However, due to problems such as easy induction of pain, skin infections and allergic reactions, and unsatisfactory patient compliance, the development of new drug delivery approaches, in replacement of injection administration, has always been an important way to innovate new drugs. As an example, liraglutide, a hypoglycemic drug in intervention treatment of diabetes, is required to be injected once a day at present, which brings considerable inconvenience and pain to patients. Semaglutide, developed by Novo Nordisk®, has similar efficacy, and the injection frequency has been reduced to once a week. However, the troubles caused by injection administration still exist, especially for patients with chronic diseases who require long-term monitoring and repeated administration. Therefore, the oral delivery of biological drugs has important medical significance.

However, the oral bioavailability of biologic drugs is low. The underlying reasons can be mainly attributed to three major physiological barriers in a drug delivery process. First, after oral administration, drugs must pass through a physicochemical barrier posed by the gastrointestinal tract's acidic and alkaline milieu together with a spectrum of digestive enzymes. Biologic drugs such as peptides, proteins, and nucleic acids are prone to denaturation, inactivation, or degradation, compromising their therapeutic efficacy. Second, after entering the gastrointestinal tract, the mucus layer constitutes a second drug absorption barrier. The mucus layer, as a dynamic gel network composed of mucins and highly glycosylated glycoproteins, blocks the invasion of pathogens through electrostatic interactions and molecular networks, but it simultaneously impedes the absorption and utilization of most macromolecular drugs. Finally, there is the absorption barrier of the small intestinal epithelial cells: because of their large size and the lack of corresponding receptors, biologic macromolecules generally fail to cross into the bloodstream via either transcellular transport or paracellular transport.

To address the above-described challenges in the oral administration of biologic drugs, pharmaceutical scientists are exploring a variety of solutions, including pH regulators, enzyme inhibitors, penetration enhancers, cell-penetrating peptides, to protect macromolecular drugs from degradation and promote their absorption and utilization. As an example, the penetration enhancers are essentially surfactants that promote drug absorption by temporarily disrupting small intestinal epithelial cells. For the semaglutide oral tablets developed by Novo Nordisk® in 2019, sodium 8-(2-hydroxybenzamido)octanoate (salcaprozate sodium) used is a typical penetration enhancer, which is tightly bound to semaglutide through non-covalent interactions, to effectively enhance the lipophilicity of the drug, thereby promoting absorption of semaglutide by epithelial cells. By introducing various functional ingredients, the stability and absorption efficiency of drug can be improved. However, due to the need to consider biosafety, the above-described methods often only achieve a bioavailability of approximately 1%. In addition, due to differences in the chemical structure, hydrophilicity-hydrophobicity, and degradation rate of different drugs, the types and proportions of adjuvants are often not universal, resulting in a long research and development cycle. New oral drug delivery technologies such as intestinal patches, gastrointestinal hydrogels, and external field-controlled drug delivery apparatuses are still difficult to pass through the above-described three physiological barriers and face problems such as complex operations and poor biocompatibility. Therefore, relevant technologies need to be further developed. Currently, effective and universal oral delivery technologies for biologic drugs are rarely available.

The microneedle drug delivery apparatus for the gastrointestinal wall is a highly promising oral drug delivery apparatus proposed in recent years. As a physical drug delivery approach, microneedles can effectively penetrate the mucus layer and epithelial cells, delivering drugs directly to the gastrointestinal wall tissue and allowing absorption of the drugs through capillaries, regardless of the molecular weight of the loaded drug. Therefore, the apparatus has versatility and effectiveness in drug delivery. In addition, since the intestinal wall lacks pain-sensing nerves, the microneedles piercing the intestinal wall will not cause pain to the patient, and intestinal perforation can be avoided by controlling the size of the microneedles. Therefore, compared with other oral drug delivery approaches, this type of microneedle drug delivery apparatus is expected to achieve relatively high drug bioavailability on the premise of safety.

One type of publicly reported microneedle drug delivery technology for gastrointestinal tract requires the pre-placement of springs, elastomers, or reactive chemicals in the apparatus. These components drive the drug-loaded microneedles to penetrate the gastrointestinal wall by rapidly releasing elastic potential energy or generating gas through rapid reactions. In addition, there is also a patent disclosure of a type of an apparatus loaded with external field-controlled substances. The microneedle penetration process is accomplished by controlling the apparatus through external fields such as magnetic fields. This physical penetration method ensures that biologic macromolecules are free from the restrictions of the three barriers in the organism and directly enter the bloodstream, thereby improving the oral bioavailability of biologic drugs. The above-described technologies face three challenges. First, biosafety is poor. These technologies employ rigid materials such as springs, elastomers, and magnetically sensitive substances as driving units, which are difficult to degrade and cause harm due to long-term exposure or retention in the gastrointestinal tract. At the same time, the patient acceptance is low and compliance is poor. Second, the success rate of drug delivery is difficult to guarantee. The one-time rapid propulsion method represented by springs can hardly guarantee a consistent success rate when facing the physiological differences of different patients. Third, the retention effect of microneedles is neglected. Existing microneedle drug delivery apparatuses mainly focus on the process of penetrating the epithelial cells of gastrointestinal wall, neglecting the retention effect of microneedles in the gastrointestinal wall. Microneedles may fall out of the gastrointestinal wall along with gastrointestinal peristalsis, which will affect the drug delivery efficiency.

Therefore, it is urgent to develop a drug delivery apparatus that can be taken orally and has high drug utilization.

### SUMMARY

The present disclosure aims to solve at least one of the technical problems in the related art to a certain extent. To this end, an objective of the present disclosure is to provide a microneedle drug delivery apparatus and a preparation method thereof. The microneedle drug delivery apparatus provided in the present disclosure allows microneedles to penetrate an intestinal wall by utilizing a mild water-absorbing swellable mechanism and natural peristaltic behavior of a gastrointestinal tract, to complete drug delivery. The operation thereof is simple and a driving mode thereof is mild, without requiring external field-controlled device or non-degradable rigid driving units, thereby having high safety. Meanwhile, a safe and effective range is provided based on an action mode of intestinal contraction force, making technical solutions provided in the present disclosure more universal.

To this end, an aspect of the present disclosure provides a microneedle drug delivery apparatus. According to embodiments of the present disclosure, the microneedle drug delivery apparatus includes: a substrate; an array of microneedles loaded on the substrate, the microneedles carrying a drug at tips of the microneedles; and a liquid-absorbing swellable material wrapped by the substrate to form a closed sandwich structure.

According to the embodiments of the present disclosure, the drug includes at least one selected from a biologic macromolecular drug and a chemical small-molecule drug.

According to the embodiments of the present disclosure, the biologic macromolecular drug includes at least one selected from a peptide drug, a protein drug, a glycan drug, and a nucleic acid drug.

According to the embodiments of the present disclosure, the microneedles contained in the array of microneedles have each a monolayer conical shape or a barbed structure. The number of the microneedles ranges from 1 to 1,000.

According to the embodiments of the present disclosure, the barbed structure includes at least one selected from a pine tree-like shape, a tower-like shape, or a thorn-like shape.

According to the embodiments of the present disclosure, the microneedles contained in the array of microneedles have each a length ranging from 30 μm to 5,000 μm, a bottom dimension ranging from 10 μm to 5,000μm, and a tip dimension ranging from 1 μm to 300 μm. The bottom dimension is greater than or equal to the tip dimension.

According to the embodiments of the present disclosure, the substrate is a flexible and stretchable substrate. The substrate is made of a material including at least one selected from sodium alginate, chitosan, cellulose, and polyvinyl alcohol.

According to the embodiments of the present disclosure, a surface of the substrate has a structure of micropores.

According to the embodiments of the present disclosure, the micropores have each a diameter ranging from 10 μm to 1,000 μm.

According to the embodiments of the present disclosure, the micropores have each a diameter ranging from 80 μm to 500 μm.

According to the embodiments of the present disclosure, a density of the micropores ranges from 0.01/mm² to 1,000/mm².

According to the embodiments of the present disclosure, the liquid-absorbing swellable material includes at least one selected from a water-absorbing gel, a water-absorbing rubber, and a water-absorbing resin.

According to the embodiments of the present disclosure, the liquid-absorbing swellable material is selected from a water-absorbing polymeric resin.

According to the embodiments of the present disclosure, the water-absorbing polymeric resin has a particle size ranging from 0.01 mm to 20 mm.

According to the embodiments of the present disclosure, the water-absorbing polymeric resin has a particle size ranging from 0.5 mm to 5 mm.

According to the embodiments of the present disclosure, the microneedle drug delivery apparatus further includes a controllable exposure device. The substrate, the array of microneedles, and the liquid-absorbing swellable material are encapsulated in the controllable exposure device.

According to the embodiments of the present disclosure, the controllable exposure device includes an enteric coating.

According to the embodiments of the present disclosure, a dimension of the microneedle drug delivery apparatus at swelling equilibrium does not exceed an inner diameter of an intestinal lumen of a subject.

Another aspect of the present disclosure provides a method for preparing the above-described microneedle drug delivery apparatus. The method includes: step 1 of preparing an array of microneedles; step 2 of preparing a substrate, and loading the array of microneedles obtained in step 1 onto a surface of the substrate to obtain a substrate-microneedle array structure; and step 3 of preparing a liquid-absorbing swellable material, and placing the liquid-absorbing swellable material inside the substrate of the substrate-microneedle array structure obtained in step 2 to form a closed sandwich structure.

According to the embodiments of the present disclosure, in step 1, the array of microneedles is prepared by at least one selected from 3D printing, photolithography, soft lithography, thermoplastic extrusion stretching, laser cutting, and casting.

According to the embodiments of the present disclosure, said preparing the array of microneedles in step 1 includes: mixing the drug with a polymer solution 1, filling the mixture into a needle tip position of a mold, and performing photo-crosslinking to obtain the array of microneedles. A polymer of the polymer solution 1 includes at least one selected from methacrylated gelatin, methacrylated dextran, methacrylated sodium alginate, methacrylated hyaluronic acid, polyethylene glycol diacrylate, or polyethylene glycol.

According to the embodiments of the present disclosure, in step 2, the substrate is prepared by at least one selected from spin coating, blade coating, photo-crosslinking, chemical crosslinking, physical crosslinking, or freeze-thaw cycling.

According to the embodiments of the present disclosure, in step 2, the array of microneedles is loaded onto the surface of the substrate by at least one selected from photo-crosslinking, chemical crosslinking, or physical attachment.

According to the embodiments of the present disclosure, said preparing the substrate in step 2 includes: placing a polymer solution 2 into a mold, performing photo-crosslinking on the polymer solution 2 and the array of microneedles obtained in step 1, and demolding, to obtain the substrate-microneedle array structure. A polymer in the polymer solution 2 includes at least one selected from a polymer with a photo-crosslinking group, a polymer monomer molecule with a photo-crosslinking group, and a composite of a polymer without a photo-crosslinking group and a polymer monomer molecule with a photo-crosslinking group.

According to the embodiments of the present disclosure, the polymer with a photo-crosslinking group includes at least one selected from methacrylated gelatin, methacrylated chitosan, polyether F127 acrylate, or polyethylene glycol acrylate. The polymer without a photo-crosslinking group includes at least one selected from polyvinyl alcohol, sodium alginate, chitosan, or cellulose. The polymer monomer molecule with a photo-crosslinking group includes at least one selected from acrylamide, acrylic acid, N,N-methylene acrylamide, or methyl acrylate.

According to the embodiments of the present disclosure, step 3 further includes: preparing a natural water-absorbing polymeric resin, placing the natural water-absorbing polymeric resin inside the substrate of the substrate-microneedle array structure prepared in step 2 to form the closed sandwich structure, to obtain the microneedle drug delivery apparatus.

According to the embodiments of the present disclosure, the drug includes at least one selected from a biologic macromolecular drug and a chemical small-molecule drug.

According to the embodiments of the present disclosure, the biologic macromolecular drug includes at least one selected from a peptide drug, a protein drug, a glycan drug, and a nucleic acid drug.

According to the embodiments of the present disclosure, said photo-crosslinking in step 1 includes: mixing the drug, the polymer solution 1, and a photoinitiator 1 to obtain a photo-crosslinking solution; dropwise adding the photo-crosslinking solution into the needle tip position of the mold; filling the photo-crosslinking solution at the needle tip position sufficiently, by means of ultrasound, centrifugation, or evacuation; and performing crosslinking.

According to the embodiments of the present disclosure, the photoinitiator 1 includes at least one selected from benzoin and derivatives thereof, benzoyl compounds, alkyl phenones, acylphosphine oxides, or benzophenones.

According to the embodiments of the present disclosure, said photo-crosslinking in step 2 includes: mixing the polymer solution 2 with a photoinitiator 2; placing the mixture in a mold; and performing ultraviolet crosslinking in an ice-water bath.

According to the embodiments of the present disclosure, the photoinitiator 2 includes at least one selected from benzoin and derivatives thereof, benzoyl compounds, alkyl phenones, acylphosphine oxides, or benzophenones.

According to the embodiments of the present disclosure, step 2 further includes, subsequent to said photo-crosslinking: performing a freeze-thaw cycling treatment.

According to the embodiments of the present disclosure, in step 3, the natural water-absorbing polymeric resin is placed in a cavity formed by the substrate, and the natural water-absorbing polymeric resin is connected to the substrate using biologic glue or by means of hot pressing.

According to the embodiments of the present disclosure, a raw material for preparing the liquid-absorbing swellable material in step 3 includes a water-absorbing polymer and a crosslinking agent.

According to the embodiments of the present disclosure, the water-absorbing polymer includes at least one selected from sodium carboxymethyl cellulose, carboxymethyl chitosan, sodium carboxymethyl alginate, or hyaluronic acid. The crosslinking agent includes at least one selected from citric acid, 1-ethyl-(3-dimethylaminopropyl)carbodiimide, glutaraldehyde, or genipin.

Another aspect of the present disclosure provides use of the above-described microneedle drug delivery apparatus in the manufacture of a medicament. The medicament is administered orally.

Yet another aspect of the present disclosure provides a medicament. The medicament includes the above-described microneedle drug delivery apparatus and an active pharmaceutical ingredient.

Yet still another aspect of the present disclosure provides a method for preparing a medicament. The method includes: placing an active pharmaceutical ingredient into microneedles of the above-described microneedle drug delivery apparatus or into microneedles of a microneedle drug delivery apparatus prepared by the above-described method.

The present disclosure has the following beneficial effects compared with the related art:
(1) Compared with the existing pharmaceutical technology, the present disclosure enables a physiological barrier of oral delivery and absorption of biologic drugs by physical means, improving oral bioavailability of the biologic drugs.
(2) Compared with the existing gastrointestinal delivery apparatuses, a driving module of the drug delivery apparatus of the present disclosure does not use a non-degradable rigid object of metallic or rubber-like material, such as a spring, an elastomer, and a magnetically sensitive particle. All materials used are biodegradable soft materials, which will not remain in the human body for a long time period or block the digestive tract, thereby having high safety and good patient compliance.
(3) According to the present disclosure, one-time trigger-type delivery mode and external field-controlled delivery mode are avoided. Instead, the microneedles pierce the intestinal wall by utilizing the volume effect of the swollen apparatus and peristaltic contraction force of the intestinal tract itself to complete the delivery, merely requiring simple and natural operation as well as a mild driving mode.
(4) According to the present disclosure, the retention effect of the microneedles in the intestinal wall tissue is improved through a barbed microneedle structure, preventing the microneedles that have penetrated the intestinal wall from falling off during intestinal peristalsis.
(5) According to the present disclosure, a drug release rate can be adjusted by regulating a degree of crosslinking, and the swelling equilibrium volume can be controlled by adjusting a filler amount of the liquid-absorbing swellable material and a mechanical strength of the flexible and stretchable substrate, which are convenient and controllable. In addition, according to the present disclosure, the drug-loaded microneedles can be driven by the normal intestinal contraction movement to pierce the intestinal wall, and a safe and effective range of swelling equilibrium volume can be set based on the action mode of intestinal contraction force, enabling the apparatus provided by the present disclosure to be more universal.

Additional aspects and advantages of the present disclosure will be provided at least in part in the following description, or will become apparent at least in part from the following description, or can be learned from practicing of the present disclosure.

### BRIEF DESCRIPTION OF THE DRAWINGS

The above and/or additional aspects and advantages of the present disclosure will become more apparent and more understandable from the following description of embodiments taken in conjunction with the accompanying drawings.

FIG. 1 to FIG. 7 are each a schematic diagram for illustrating an appearance of a microneedle drug delivery apparatus according to the present disclosure. FIG. 1 to FIG. 3 are schematic diagrams for illustrating appearances of a spherical microneedle drug delivery apparatus, a cylindrical microneedle drug delivery apparatus, and a capsule-type microneedle drug delivery apparatus according to the present disclosure, respectively. FIG. 4 to FIG. 6 are schematic diagrams for illustrating appearances of a spherical microneedle drug delivery apparatus, a cylindrical microneedle drug delivery apparatus, and a capsule-type microneedle drug delivery apparatus according to the present disclosure after absorbing water and swelling, respectively. FIG. 7 is a schematic diagram for illustrating a longitudinal cross-section of a capsule-type microneedle drug delivery apparatus of the present disclosure before and after absorbing water and swelling.

FIG. 8 illustrates a mold for manufacturing an array of microneedles in the present disclosure.

FIG. 9 is a preparation flowchart of the substrate-microneedle array structure in Example 1 of the present disclosure, where: Figure a is a preparation flowchart of a substrate-conical microneedle array structure; and Figure b is a preparation flowchart of a substrate-pine tree-shaped or tower-shaped microneedle array structure.

FIG. 10 is an outside view of the microneedle delivery apparatus loaded in a commercial capsule shell in Example 2 of the present disclosure.

FIG. 11 is a diagram of pressure applied by the intestinal tract on sensors of different sizes in Example 3 of the present disclosure.

FIG. 12 is a diagram showing results of pressure and force exerted by the intestinal tract on the microneedles in Example 3 of the present disclosure.

FIG. 13 and FIG. 14 reveal hypoglycemic effects of insulin-loaded microneedle drug delivery apparatus on experimental miniature pigs in Example 4 of the present disclosure.

FIG. 15 reveals drug delivery effects of the insulin-loaded microneedle drug delivery apparatus in Example 4 of the present disclosure.

FIG. 16 reveals the comparison of areas under the pharmacokinetic curve for insulin delivery through the intestinal delivery, subcutaneous injection, intestinal delivery via the conical microneedle drug delivery apparatus, and intestinal delivery via the tower-shaped microneedle drug delivery apparatus in Example 4 of the present disclosure.

FIG. 17 reveals gastroenteroscopic images of a microneedle drug delivery apparatus in the small intestine in Example 5 of the present disclosure.

FIG. 18 reveals small intestine section images of in vivo intestinal tract of experimental miniature pigs, in vitro intestinal tract of experimental miniature pigs, and a blank control group, after the microneedle drug delivery apparatus in Example 5 of the present disclosure exerted its effects.

### DETAILED DESCRIPTION

Embodiments of the present disclosure will be described in detail below. The embodiments described below are exemplary and are only used to explain the present disclosure, and they should not be construed as limiting the present disclosure.

It should be noted that the terms “first” and “second” are only used for descriptive purposes, rather than indicating or implying relative importance or implicitly indicating the number of indicated technical features. Therefore, the features defined with “first” and “second” may include one or more of these features explicitly or implicitly. In the description of the present disclosure, “a plurality of” means at least two, for example, two or three, unless specified otherwise.

To make the present disclosure more easily understood, certain technical and scientific terms are specifically defined below. Unless otherwise clearly defined, all other technical and scientific terms used herein have the meanings commonly understood by those skilled in the art to which the present disclosure belongs.

As used herein, the term “comprise” or “include” is an open expression, that is, including the contents indicated by the present disclosure, but not excluding other aspects.

The term “peptide” refers to a compound formed by linking α-amino acids together by peptide bonds, and is an intermediate product of protein hydrolysis. A compound formed by the dehydration condensation of two amino acid molecules is called a dipeptide; similarly, there are also tripeptides, tetrapeptides, pentapeptides, etc.

The term “peptide” refers to a peptide composed of three or more amino acid molecules. In the present disclosure, when the drug encapsulated in the microneedles is a peptide drug, it includes, but is not limited to, antibody drugs.

The term “small-molecule drug” refers to a chemically synthesized small-molecule drug, typically with a relative molecular mass below 1000.

The term “dimension of the microneedle drug delivery apparatus at swelling equilibrium” refers to the dimension of the microneedle drug delivery apparatus when the gradually decreasing swelling force and the gradually increasing substrate tension reach the equilibrium during a swelling process of the microneedle drug delivery apparatus.

The term “area under pharmacokinetic curve (AUC)” refers to the average area under the plasma drug concentration-time curve calculated using the trapezoid rule over the entire sample collection interval.

According to a specific embodiment of the present disclosure, an aspect of the present disclosure provides a microneedle drug delivery apparatus. The apparatus includes a substrate, an array of microneedles, and a liquid-absorbing swellable material. The array of microneedles is supported on the substrate. The liquid-absorbing swellable material is wrapped by the substrate to form a closed sandwich structure. The microneedles carry a drug at tips of the microneedles.

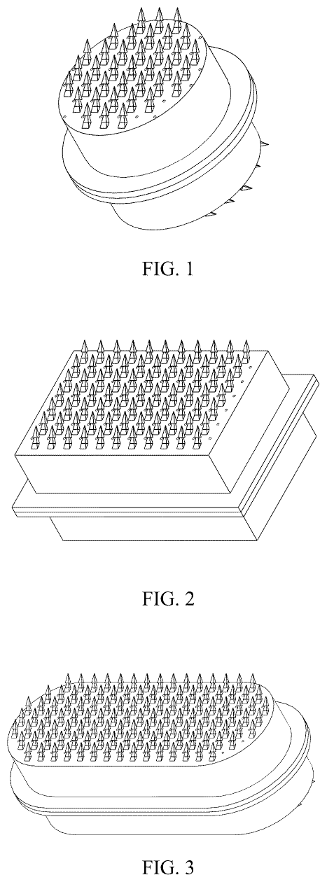

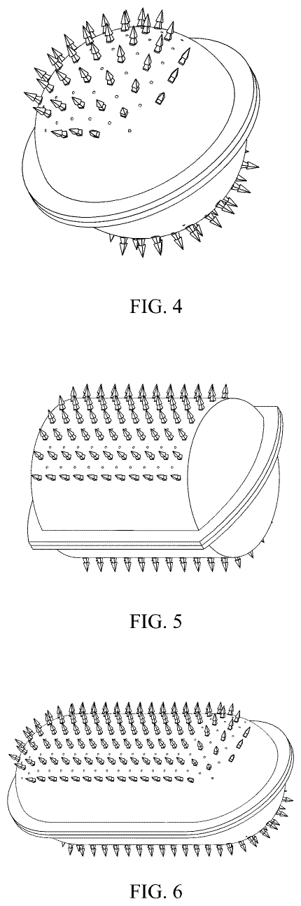

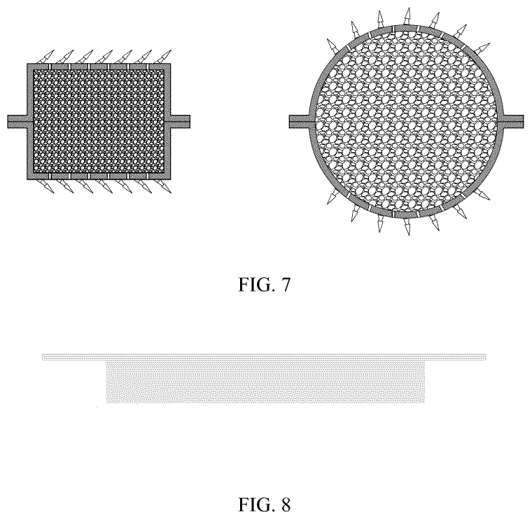

Since the liquid-absorbing swellable material has the property of swelling upon solvent absorption, when the microneedles inside the apparatus lie prostrate in the dry state and thus can withstand compression of the gastrointestinal tract. Once external digestive fluid or water penetrates the apparatus, the liquid-absorbing swellable material absorbs the liquid and swells, the substrate stretches, and the microneedles anchored on the substrate erect outward and pierce the intestinal wall by virtue of an intestinal peristalsis. This design minimizes the influence of gastric mobility on the microneedles and prevents the array of microneedles from breaking or falling off before reaching the intestinal tract. As illustrated in FIG. 1 to FIG. 7, FIG. 1 to FIG. 3 are each a schematic diagrams for illustrating an appearance of a microneedle drug delivery apparatus provided in the present disclosure, which may be spherical, rectangular, capsule-type, or any other geometry; FIG. 4 to 6 are each a schematic diagrams for illustrating an appearance of a microneedle drug delivery apparatus prepared according to the present disclosure after absorbing water and swelling; and FIG. 7 is a schematic diagram for illustrating a longitudinal cross-section of a capsule-type microneedle drug delivery apparatus before and after absorbing water and swelling. Assuming constant frequency and amplitude of small-intestine peristaltic contractions, a larger volume of the microneedle drug delivery apparatus experiences greater force. However, because intestinal tissue is compliant and can conform to the apparatus contour during contraction, the microneedle drug delivery apparatus provided in the present disclosure is not restricted to cylindrical, spherical, or capsule shapes. If the radial compressive pressure exerted by the intestinal wall is uniform, a higher apparent compressive modulus and strength of the apparatus will yield smaller deformation under peristaltic squeeze, the apparatus is subjected greater force, which facilitates microneedle piercing the intestinal wall. Moreover, since the drug-loaded microneedle material does not materially affect the drug itself, the disclosed device can encapsulate any desired therapeutic agent.

According to a specific embodiment of the present disclosure, the drug includes at least one selected from a biologic macromolecular drug and a chemical small-molecule drug.

According to a specific embodiment of the present disclosure, the biologic macromolecular drug includes at least one selected from a peptide drug, a protein drug, a glycan drug, and a nucleic acid drug. The microneedle drug delivery apparatus can encapsulate, but not limited to, the peptide, the protein, the glycan, and the nucleic acid drug.

According to a specific embodiment of the present disclosure, the microneedles contained in the array of microneedles have each a monolayer conical shape or a barbed structure. The number of the microneedles ranges from 1 to 1,000.

According to a specific embodiment of the present disclosure, the barbed structure includes at least one selected from a pine tree-like shape, a tower-like shape, or a thorn-like shape.

By designing the microneedles to have a conical, pine tree-like, tower-like, or thorn-like structure, the force required to detach the microneedles that have penetrated the intestinal wall can be increased, and thus a retention effect of the microneedles in the intestinal wall can be improved, improving bioavailability of the drug.

According to a specific embodiment of the present disclosure, the microneedles contained in the array of microneedles have each a length ranging from 30 μm to 5,000 μm, a bottom dimension ranging from 10 μm to 5,000 μm, and a tip dimension ranging from 1 μm to 300 μm. The bottom dimension is greater than or equal to the tip dimension.

According to a specific embodiment of the present disclosure, the substrate is a flexible and stretchable substrate. The substrate is made of a material including at least one selected from sodium alginate, chitosan, cellulose, and polyvinyl alcohol. The flexible and stretchable substrate can be stretched and extended, to change the shape of the microneedle drug delivery apparatus in the intestinal tract in combination with the property of swelling upon solvent absorption of the liquid-absorbing swellable material. When the microneedle drug delivery apparatus is squeezed by the intestinal tract, assuming that the volume of the liquid-absorbing swellable material remains unchanged, the intestinal compressive force is mainly balanced by tension generated by further extension of a flexible and stretchable membrane. Therefore, a flexible and stretchable membrane with high toughness and fatigue resistance is required to withstand frequent contraction movement of the intestinal wall and maintain integrity of the apparatus in an early stage.

According to a specific embodiment of the present disclosure, a surface of the substrate has a structure of micropores. The structure of micropores can be constructed using pore-forming methods such as needle punching and laser drilling, and the micropores serve as water-absorbing channels to facilitate the entry of external digestive fluids or water into the interior of the apparatus.

According to a specific embodiment of the present disclosure, the micropores have each a diameter ranging from 10 μm to 1,000 μm.

According to a preferred embodiment of the present disclosure, the micropores have each a diameter ranging from 80 μm to 500 μm.

According to a specific embodiment of the present disclosure, a density of the micropores ranges from 0.01/mm² to 1,000/mm².

According to a specific embodiment of the present disclosure, the liquid-absorbing swellable material includes at least one selected from a water-absorbing gel, a water-absorbing rubber, and a water-absorbing resin.

According to a specific embodiment of the present disclosure, the liquid-absorbing swellable material is selected from a water-absorbing polymeric resin.

According to a specific embodiment of the present disclosure, the water-absorbing polymeric resin has a particle size ranging from 0.01 mm to 20 mm.

According to a preferred embodiment of the present disclosure, the water-absorbing polymeric resin has a particle size ranging from 0.5 mm to 5 mm.

According to a specific embodiment of the present disclosure, the microneedle drug delivery apparatus further includes a controllable exposure device. The substrate, the array of microneedles, and the liquid-absorbing swellable material are encapsulated in the controllable exposure device.

According to a specific embodiment of the present disclosure, the controllable exposure device includes an enteric coating.

The above-described enteric coating can be prepared through spray coating and dip coating. Specifically, the spraying coating involves: preparing a 95% ethanol solution containing 6% Eudragit L-100, 0.6% triethyl citrate, and 1.25% talc; spraying micron-sized droplets onto the controlled exposure device using a spray gun under appropriate air pressure and liquid discharge rate; and after the solvent has fully evaporated, repeating the above operations multiple times, to form the enteric coating. The dipping coating involves: preparing an ethanol solution containing 7% Eudragit L-100; immersing the controlled exposure device in this solution; and after the solvent has fully evaporated, repeating the above operations multiple times, to form the enteric coating.

According to a specific embodiment of the present disclosure, a dimension of the microneedle drug delivery apparatus at swelling equilibrium does not exceed an inner diameter of an intestinal lumen of a subject, which allows the apparatus to deform and move under axial pressure, avoiding intestinal obstruction. The dimension of the apparatus at the swelling equilibrium is crucial for utilizing the radial compressive force generated during small intestinal peristalsis. Assuming that a radial compressive force exerted by the small intestinal wall is uniform, a higher apparent compressive modulus and strength of the apparatus will yield smaller deformation under peristaltic squeeze, the apparatus is subjected greater force, which facilitates microneedle piercing the intestinal wall. In addition, the filling amount of the liquid-absorbing swellable material and tensile properties of the substrate affect the dimension of the apparatus at the swelling equilibrium.

Another aspect of the present disclosure provides a method for preparing the above-described microneedle drug delivery apparatus. The method incudes: step 1 of preparing an array of microneedles; step 2 of preparing a substrate, and loading the array of microneedles obtained in step 1 onto a surface of the substrate to obtain a substrate-microneedle array structure; and step 3 of preparing a liquid-absorbing swellable material, and placing the liquid-absorbing swellable material inside the substrate of the substrate-microneedle array structure obtained in step 2 to form a closed sandwich structure.

According to a specific embodiment of the present disclosure, in step 1, the array of microneedles is prepared by at least one selected from 3D printing, photolithography, soft lithography, thermoplastic extrusion stretching, laser cutting, and casting.

According to a specific embodiment of the present disclosure, said preparing the array of microneedles in step 1 includes: mixing the drug with a polymer solution 1, filling the mixture into a needle tip position of a mold, and performing photo-crosslinking to obtain the array of microneedles.

A polymer of the polymer solution 1 includes at least one selected from methacrylated gelatin, methacrylated dextran, methacrylated sodium alginate, methacrylated hyaluronic acid, polyethylene glycol diacrylate, or polyethylene glycol.

According to a specific embodiment of the present disclosure, in step 2, the substrate is prepared by at least one selected from spin coating, blade coating, photo-crosslinking, chemical crosslinking, physical crosslinking, or freeze-thaw cycling.

According to a specific embodiment of the present disclosure, in step 2, the array of microneedles is loaded onto the surface of the substrate by at least one selected from photo-crosslinking, chemical crosslinking, or physical attachment.

According to a specific embodiment of the present disclosure, said preparing the substrate in step 2 includes: placing a polymer solution 2 into a mold, performing photo-crosslinking on the polymer solution 2 and the array of microneedles obtained in step 1, and demolding, to obtain the substrate-microneedle array structure.

A polymer in the polymer solution 2 includes at least one selected from a polymer with a photo-crosslinking group, a polymer monomer molecule with a photo-crosslinking group, and a composite of a polymer without a photo-crosslinking group and a polymer monomer molecule with a photo-crosslinking group.

According to a specific embodiment of the present disclosure, in step 2, the mold can be the model as illustrated in FIG. 8, including an upper cover plate and a lower concave mold that can be assembled. The lower concave mold is the needle tip position of the mold and configured to be filled with a solution for forming the microneedles, and the remaining space in the mold is configured to be filled with a solution for forming the substrate. Such a mold can be designed with the assistance of software such as Solidworks. As desired, three-dimensional models with different numbers of needles, needle lengths, needle types, microneedle densities, and microneedle tapers can be designed. A hard convex mold is obtained through rapid prototyping technology such as 3D printing, and a concave mold is obtained through PDMS molding to serve as the required mold. In addition, in the process for preparing the substrate-microneedle array structure, polymer monomer molecules are added to the pre-prepared solution for forming the substrate, to establish covalent bonds between the array of microneedles and the substrate in a co-crosslinking manner, thereby enhancing connectivity between the two components.

According to a specific embodiment of the present disclosure, the polymer with a photo-crosslinking group includes at least one selected from methacrylated gelatin, methacrylated chitosan, polyether F127 acrylate, or polyethylene glycol acrylate; the polymer without a photo-crosslinking group includes at least one selected from polyvinyl alcohol, sodium alginate, chitosan, or cellulose; and the polymer monomer molecule with a photo-crosslinking group includes at least one selected from acrylamide, acrylic acid, N,N-methylene acrylamide, or methyl acrylate.

According to a specific embodiment of the present disclosure, step 3 specifically includes: preparing a natural water-absorbing polymeric resin, placing the natural water-absorbing polymeric resin inside the substrate of the substrate-microneedle array structure prepared in step 2 to form the closed sandwich structure, to obtain the microneedle drug delivery apparatus.

According to a specific embodiment of the present disclosure, the drug includes at least one selected from a biologic macromolecular drug and a chemical small-molecule drug.

According to a specific embodiment of the present disclosure, the biologic macromolecular drug includes at least one selected from a peptide drug, a protein drug, a glycan drug, and a nucleic acid drug.

The drug can be a single pure active pharmaceutical ingredient, a mixture of multiple drugs for cocktail therapy, or a micro-nano particle containing the active pharmaceutical ingredient for enhancing sustained-release effects after drug delivery.

According to a specific embodiment of the present disclosure, said photo-crosslinking in step 1 includes: mixing the drug, the polymer solution 1, and a photoinitiator 1 to obtain a photo-crosslinking solution; adding the photo-crosslinking solution into the needle tip position of the mold dropwise; sufficiently filling the photo-crosslinking solution at the needle tip position by means of ultrasound, centrifugation, or evacuation; and performing crosslinking.

According to a specific embodiment of the present disclosure, the photoinitiator 1 includes at least one selected from benzoin and derivatives thereof, benzoyl compounds, alkyl phenones, acylphosphine oxides, or benzophenones. Benzoin and derivatives thereof may include benzoin dimethyl ether (BDK), etc.; benzoyl compounds may include methyl o-benzoyl benzoate (OMBB); alkyl phenones may include 2-hydroxy-4-(2-hydroxyethoxy)-2-methylpropiophenone (I2959), 2-hydroxy-2-methyl-1-phenyl-1-propanone (1173), 1-hydroxy-cyclohexyl-phenyl ketone (184), etc.; acylphosphine oxides may include 2,4,6-(trimethylbenzoyl)-phosphine oxide (TPO), ethyl (2,4,6-trimethylbenzoyl) phenylphosphinate (TPO-L), etc.; and benzophenones may include 4-chlorobenzophenone (CBP), 4-phenylbenzophenone (PBZ), etc.

According to a specific embodiment of the present disclosure, said photo-crosslinking in step 2 includes: mixing the polymer solution 2 with a photoinitiator 2; placing the mixture in a mold; and performing ultraviolet crosslinking in an ice-water bath.

According to a specific embodiment of the present disclosure, the photoinitiator 2 includes at least one selected from benzoin and derivatives thereof, benzoyl compounds, alkyl phenones, acylphosphine oxides, or benzophenones. Benzoin and derivatives thereof may include benzoin dimethyl ether (BDK), etc.; benzoyl compounds may include methyl o-benzoyl benzoate (OMBB); alkyl phenones may include 2-hydroxy-4-(2-hydroxyethoxy)-2-methylpropiophenone (I2959), 2-hydroxy-2-methyl-1-phenyl-1-propanone (1173), 1-hydroxy-cyclohexyl-phenyl ketone (184), etc.; acylphosphine oxides may include 2,4,6-(trimethylbenzoyl)-phosphine oxide (TPO), ethyl (2,4,6-trimethylbenzoyl) phenylphosphinate (TPO-L), etc.; and benzophenones may include 4-chlorobenzophenone (CBP), 4-phenylbenzophenone (PBZ), etc.

According to a specific embodiment of the present disclosure, step 2 further includes, subsequent to said photo-crosslinking: performing a freeze-thaw cycling treatment.

According to a specific embodiment of the present disclosure, a polyvinyl alcohol substrate is prepared through a freeze-thaw cycling method. A polyvinyl alcohol solution with a mass concentration of 5% to 15% is prepared, frozen at −20° C. for 6 hours, and then thawed at 25° C. for 30 minutes. After such a freeze-thaw cycle repeats 4 to 5 times, a polyvinyl alcohol substrate with predetermined toughness, strength, and stretchability can be obtained. In addition, the substrate obtained by this method enables formation of nanocrystals inside the molecules, facilitating energy absorption when the material deforms.

According to a specific embodiment of the present disclosure, in step 3, the natural water-absorbing polymeric resin is placed in a cavity formed by the substrate, and the natural water-absorbing polymeric resin is connected to the substrate using biologic glue or by means of hot pressing.

According to a specific embodiment of the present disclosure, a raw material for preparing the liquid-absorbing swellable material in step 3 includes a water-absorbing polymer and a crosslinking agent.

According to a specific embodiment of the present disclosure, the water-absorbing polymer includes at least one selected from sodium carboxymethyl cellulose, carboxymethyl chitosan, sodium carboxymethyl alginate, or hyaluronic acid; and the crosslinking agent includes at least one selected from citric acid, 1-ethyl-(3-dimethylaminopropyl)carbodiimide, glutaraldehyde, or genipin.

According to a specific embodiment of the present disclosure, a method for preparing sodium carboxymethyl cellulose hydrogel includes: preparing a mixed solution containing sodium carboxymethyl cellulose with a mass fraction of 1% to 10% and citric acid with a mass fraction of 0.1% to 1%, drying the solution at 50° C. for 6 hours, and annealing at 120° C. for 4 hours to complete crosslinking. When using the crosslinking agent such as 1-ethyl-(3-dimethylaminopropyl)carbodiimide, glutaraldehyde, or genipin, crosslinking can be carried out at room temperature. In addition, a swelling rate of the hydrogel itself is related to its internal micro-nano structure. A loose and porous structure can be constructed inside the hydrogel using a freeze drying method, an anti-solvent method, a salting out method, and other methods, accelerating the water absorption and swelling rate of the hydrogel.

Another aspect of the present disclosure provides use of the above-described microneedle drug delivery apparatus in the manufacture of a medicament. The medicament is administered orally.

Yet still another aspect of the present disclosure provides a medicament. The medicament includes the above-described microneedle drug delivery apparatus and an active pharmaceutical ingredient.

Yet still another aspect of the present disclosure provides a method for preparing a medicament. The method includes: placing an active pharmaceutical ingredient into microneedles of the above-described microneedle drug delivery apparatus or into microneedles of a microneedle drug delivery apparatus prepared by the above-described method.

The following examples are provided to further illustrate the solutions of the present disclosure. Those skilled in the art will understand that the following examples are merely intended to explain the present disclosure. These examples should not be construed as limitations on the scope of the present disclosure. Technical methods or conditions not specifically described in the embodiments may be implemented in accordance with techniques or conditions disclosed in the literature in the relevant field, or according to standard product manuals. Reagents or instruments without specifying the manufacturer thereof are all commercially available conventional products.

### Examples

### Example 1 Preparation of Substrate-Microneedle Array Structure

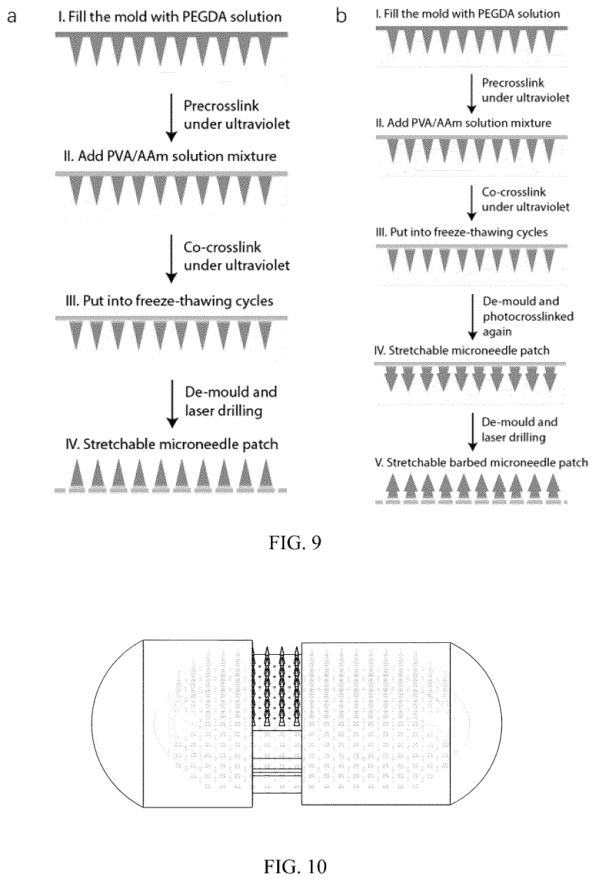

1. When the microneedles had each a conical shape, the process for preparing the substrate-microneedle array structure thereof was as illustrated in FIG. 9(a), as specifically described below.

(I) A mixed aqueous solution containing 20 mg/ml human recombinant insulin, 33% (v/v) polyethylene glycol diacrylate-600, 33% (v/v) polyethylene glycol-300, and 1% (v/v) photoinitiator 2-hydroxy-2-methylphenylacetone was prepared. 100μl of the solution was taken and added to the mold. Air bubbles were removed by means of ultrasound, centrifugation, or evacuation to fill the mixed solution at the needle holes of the microneedle mold sufficiently. Then, photocuring was performed at 365 nm for 15 seconds to obtain the array of microneedles.

(II) A mixed aqueous solution containing 8% (w/v) acrylamide, 0.1% (w/v) N, N-methyleneacrylamide, 10% polyvinyl alcohol (molecular weight between 89,000 and 98,000), and 0.1% (w/v) photoinitiator I2959 was prepared. 500 μl of the solution was taken and added to the mold, sealed with a special transparent PVC plate, and then subjected to ultraviolet crosslinking in an ice-water bath for 10 minutes to 30 minutes.

(III) The mold was subjected to 4 to 5 freeze-thaw cycles, and demolded to obtain a flexible and stretchable substrate loaded with drug-loaded microneedles.

(IV) Micropores with a diameter of 100 μm were constructed in the gaps between the microneedles on the flexible and stretchable substrate using laser drilling.

2. When the microneedles had each a pine tree-like shape or a tower-like shape, the preparation process of the substrate-microneedle array structure was illustrated in FIG. 9(b), as specifically described below.

Based on the preparation of the substrate-microneedle array structure with conical microneedles, the flexible and stretchable substrate loaded with drug-loaded microneedles obtained in step (III) was placed in a mold where the needle tip position is pine tree-shaped or tower-shaped. The needle tip position of the mold was pre-filled with a mixed solution containing a photoinitiator, drug, and a crosslinkable polymer. Photo-crosslinking was performed again to obtain microneedles with a pine tree-like structure or tower-like structure. After demolding, the flexible substrate loaded with the array of pine tree-shaped or tower-shaped microneedles was obtained. Finally, micropores with a diameter of 100 μm were constructed in the gaps between the microneedles of the flexible substrate using laser drilling. The needle tip position of the pre-prepared conical microneedles may not be filled with drugs.

### Example 2 Preparation of Microneedle Drug Delivery Apparatus Loaded in Controlled Exposure Device

(I) A 6% (w/v) sodium carboxymethyl cellulose solution was prepared, and 0.5% (w/v) 1-ethyl-(3-dimethylaminopropyl)carbodiimide was added. The mixture was stirred and crosslinked at room temperature.

(II) The crosslinked product obtained in step (I) was dried in an oven at 50° C., and then crushed using a high-speed pulverizer to obtain hydrogel particles with a particle size controlled at approximately 0.5 mm.

(III) An appropriate amount of hydrogel particles was added to the substrate loaded with the array of microneedles prepared in Example 1, and bonded using biocompatible adhesive to obtain the microneedle drug delivery apparatus. After drying the biocompatible adhesive, the apparatus was load into a Size 00 capsule and coated with an enteric coating to obtain the microneedle drug delivery apparatus as illustrated in FIG. 10.

### Example 3: Dimension Determination of Microneedle Drug Delivery Apparatus

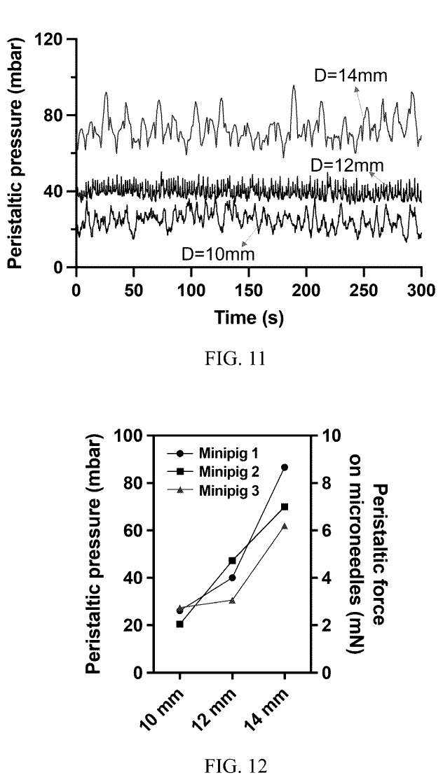

In this example, Bama minipigs were used as model animals. Capsule-type pressure sensors with diameters of 10 mm, 12 mm, and 14 mm were delivered to the small intestine using a gastrointestinal endoscope, the force exerted on the sensors with different diameters when being squeezed by the small intestine was measured, and the results were illustrated in FIG. 11. The force on each microneedle (F1) under different diameters in vivo can be calculated using the pressure reading (P) from the capsule-type sensor and the microneedle density (ρ) distributed on the drug delivery apparatus, i.e., F1=P/ρ. In addition, the force required for the microneedles to penetrate the small intestinal tissue (F2) can be measured in vitro using a force gauge. By comparing F1 and F2, the minimum diameter of the microneedle drug delivery apparatus that is required to pierce the microneedles into the intestinal wall can be obtained, and the inner diameter of the small intestine when distended was used as the maximum diameter, thereby determining the dimension range of the microneedle drug delivery apparatus. The pressure and force exerted by the intestinal tract on the microneedles are illustrated in FIG. 12.

In addition, the dimension of the microneedle drug delivery apparatus was mainly regulated by the stretchable substrate and the hydrogel filled in the substrate. That is, during the swelling process of the microneedle drug delivery apparatus, the tension (σ) of the stretchable substrate gradually increased, while the water absorption osmotic pressure (Ps) of the hydrogel gradually decreased. When these two values were equal, the dimension of the microneedle drug delivery apparatus reached equilibrium. Specifically, the expandable dimension of the microneedle drug delivery apparatus increases with a decrease of the strength of the stretchable substrate, i.e., the Young's modulus; and the expandable dimension of the microneedle drug delivery apparatus increases with an increase in the water absorption osmotic pressure and filling amount of the hydrogel. Through theoretical calculations and experimental verification, when sodium carboxymethyl cellulose water-absorbing hydrogel particles and a stretchable substrate with a filling amount of hydrogel ranging from 12.5% to 37.5% and with a Young's modulus ranging from 60 kPa to 200 kPa were selected to prepare the microneedle drug delivery apparatus, the prepared microneedle drug delivery apparatus can swell to 10 mm to 14 mm after fully absorbing water.

### Example 4: Functional Verification of Microneedle Drug Delivery Apparatus

Considering the similarities between pigs and humans in the structure of the digestive system and physiological functions, experimental minipigs were used as the animal model for functional verification of the apparatus in this example. To prevent the pigs' chewing behavior from damaging the microneedle drug delivery apparatus and to take the individual variations in gastric emptying time into account, in this example, after anesthetizing the pigs, the microneedle drug delivery apparatus prepared in Example 2 was delivered into the pigs' duodenum using a gastroscope, and this moment was defined as time zero, the beginning of monitoring of the drug concentration in serum and the drug delivery effect. Central venous blood samples were collected every 20 minutes for a continuous time period of 4 hours.

### (1) Hypoglycemic Effect

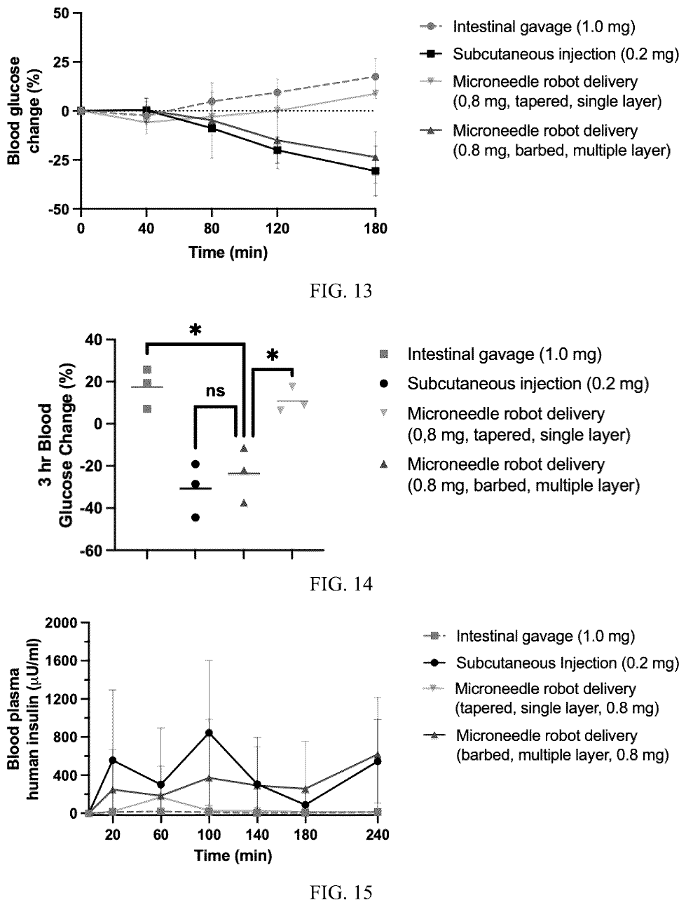

Real-time blood glucose was measured using a portable blood glucose meter, and the results were illustrated in FIG. 13 and FIG. 14. Through comparison, it can be found that when insulin was delivered via the intestinal tract, the hypoglycemic effect was only achieved for a short time period, and the blood glucose level in the blood still rose afterward, resulting in a poor hypoglycemic effect. In contrast, when the insulin was delivered using the microneedle drug delivery apparatus prepared according to the present disclosure, its hypoglycemic effect was superior to that of intestinal delivery. The microneedle drug delivery apparatus with a barbed structure achieved an effect comparable to that achieved by subcutaneous injection, reducing blood glucose by 23.7% after 3 hours.

### (2) Bioavailability

Excess blood was placed in a coagulation tube, left to stand for approximately 40 minutes, and then centrifuged to separate the serum. The drug content in the blood was detected using a human insulin ELISA kit, yielding the pharmacokinetic curve as shown in FIG. 15. Due to differences in insulin sensitivity among individual animals, variations in intestinal peristalsis rhythms across animals, and inconsistent squeezing patterns on the microneedle drug delivery apparatus, the overall results exhibited differences in the time points of peak plasma concentration and large error bars at each data point. Calculating the area under this curve (AUC) can be used to compare the bioavailability of different drug delivery methods.

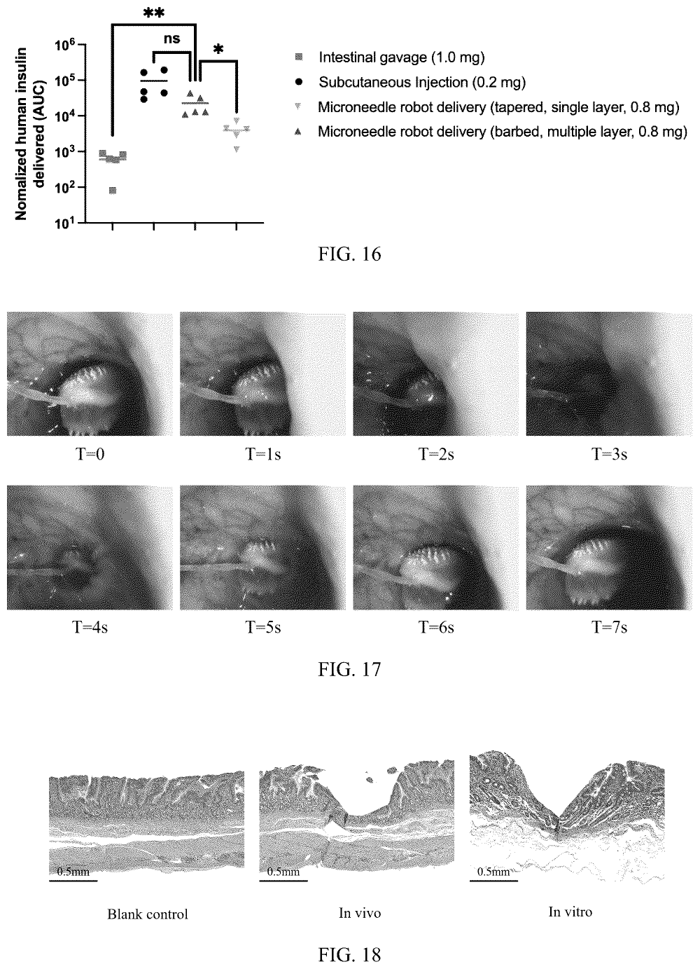

Bioavailability referred to the relative amount of a drug that was absorbed and entered the systemic circulation after administration via an extravascular route. The bioavailability was calculated as the ratio of the area under the pharmacokinetic curve (AUC)/administered dose of the test group to the corresponding value of the subcutaneous injection group, for evaluating the efficiency of a drug delivery method. FIG. 16 was obtained by statistically analyzing the AUC of each curve in FIG. 15, and the bioavailability of each drug delivery method was calculated using the formula (AUC with barbs/D)/(AUC Sub/0.2)×100%. The intestinal delivery, the tower-shaped microneedle drug delivery apparatus, and the conical drug delivery apparatus have a bioavailability of 0.6%, 23.6%, and 4.2%, respectively, indicating that the microneedle drug delivery apparatus prepared according to the present disclosure, i.e., either the tower-shaped microneedle drug delivery apparatus or the conical microneedle drug delivery apparatus, can achieve relatively high bioavailability when delivering drugs.

### Example 5: Biologic Verification of Microneedle Drug Delivery Apparatus

Experimental Bama minipigs were used as model animals, and the microneedle drug delivery apparatus was delivered to the small intestine using a gastrointestinal endoscope. Continuous observation using the endoscope, the results in FIG. 17 reveal that the microneedle drug delivery apparatus was subjected to sufficient squeezing in the intestinal tract after swelling.

After waiting for 4 hours to allow the microneedle drug delivery apparatus to exert its effect, the abdomen of the Bama minipig was dissected, the intestinal tract was taken out to identify the location of the microneedle drug delivery apparatus, and the small intestinal wall adjacent to the location of the microneedle drug delivery apparatus was excised. In addition, the microneedle drug delivery apparatus was used to directly pierce a piece of small intestinal wall in vitro. These two pieces of small intestinal wall were fixed by immersion in formalin, underwent dehydration and paraffin embedding, and were subsequently sectioned and subjected to HE staining for observation. The results as illustrated in FIG. 18 clearly revealed microneedle piercing tracks in both the in-vivo and ex-vivo intestinal walls, whereas no such tracks were found on the small intestinal sections of the blank control group, indicating that the microneedle drug delivery apparatus is capable of piercing the microneedles into the intestinal wall.

Reference throughout the specification to “an embodiment”, “some embodiments”, “an illustrative embodiment”, “an example”, “a specific example”, or “some examples” means that a particular feature, structure, material, or characteristic described in connection with the embodiment or example is included in at least one embodiment or example of the present disclosure. The appearances of the above phrases in various places throughout this specification are not necessarily referring to the same embodiment or example. Further, the particular features, structures, materials, or characteristics may be combined in any suitable manner in one or more embodiments or examples. In addition, those skilled in the art can combine the different embodiments or examples and the features of the different embodiments or examples described in this specification without contradicting each other.

Although embodiments of the present disclosure have been illustrated and described above, it should be understood that the above embodiments are merely exemplary, and cannot be construed to limit the present disclosure. For those skilled in the art, changes, modifications, substitutions, and variations can be made to the embodiments without departing from the scope of the present disclosure.

What is claimed is:

1. A microneedle drug delivery apparatus, comprising:
a substrate;
an array of microneedles loaded on the substrate, the microneedles carrying a drug at tips of the microneedles; and
a liquid-absorbing swellable material wrapped by the substrate to form a closed sandwich structure.

2. The microneedle drug delivery apparatus according to claim 1, wherein the drug comprises at least one selected from a biologic macromolecular drug and a chemical small-molecule drug,
optionally, the biologic macromolecular drug comprises at least one selected from a peptide drug, a protein drug, a glycan drug, and a nucleic acid drug.

3. The microneedle drug delivery apparatus according to claim 1, wherein:
the microneedles contained in the array of microneedles have each a monolayer conical shape or a barbed structure; and
the number of the microneedles ranges from 1 to 1,000.

4. The microneedle drug delivery apparatus according to claim 3, wherein the barbed structure comprises at least one selected from a pine tree-like shape, a tower-like shape, or a thorn-like shape.

5. The microneedle drug delivery apparatus according to claim 1, wherein the microneedles contained in the array of microneedles have each a length ranging from 30 μm to 5,000 μm, a bottom dimension ranging from 10 μm to 5,000 μm, and a tip dimension ranging from 1 μm to 300 μm, the bottom dimension being greater than or equal to the tip dimension.

6. The microneedle drug delivery apparatus according to claim 1, wherein:
the substrate is a flexible and stretchable substrate; and
the substrate is made of a material comprising at least one selected from sodium alginate, chitosan, cellulose, and polyvinyl alcohol.

7. The microneedle drug delivery apparatus according to claim 1, wherein a surface of the substrate has a structure of micropores, wherein the micropores have each a diameter ranging from 10 μm to 1,000 μm, and a density of the micropores ranges from 0.01/mm² to 1,000/mm².

8. The microneedle drug delivery apparatus according to claim 1, wherein the liquid-absorbing swellable material comprises at least one selected from a water-absorbing gel, a water-absorbing rubber, and a water-absorbing resin.

9. The microneedle drug delivery apparatus according to claim 1, wherein the liquid-absorbing swellable material is selected from a water-absorbing polymeric resin, wherein the water-absorbing polymeric resin has a particle size ranging from 0.01 mm to 20 mm.

10. The microneedle drug delivery apparatus according to claim 1, further comprising a controllable exposure device, wherein the substrate, the array of microneedles, and the liquid-absorbing swellable material are encapsulated in the controllable exposure device, and wherein the controllable exposure device comprises an enteric coating.

11. The microneedle drug delivery apparatus according to claim 1, wherein a dimension of the microneedle drug delivery apparatus at swelling equilibrium does not exceed an inner diameter of an intestinal lumen of a subject.

12. A method for preparing the microneedle drug delivery apparatus according to claim 1, the method comprising:
step 1 of preparing an array of microneedles;
step 2 of preparing a substrate, and loading the array of microneedles obtained in step 1 onto a surface of the substrate to obtain a substrate-microneedle array structure; and
step 3 of preparing a liquid-absorbing swellable material, and placing the liquid-absorbing swellable material inside the substrate of the substrate-microneedle array structure obtained in step 2 to form a closed sandwich structure.

13. The method according to claim 12, wherein said preparing the array of microneedles in step 1 comprises: mixing the drug with a polymer solution 1, filling the mixture into a needle tip position of a mold, and performing photo-crosslinking to obtain the array of microneedles,
wherein a polymer of the polymer solution 1 comprises at least one selected from methacrylated gelatin, methacrylated dextran, methacrylated sodium alginate, methacrylated hyaluronic acid, polyethylene glycol diacrylate, or polyethylene glycol.

14. The method according to claim 12, wherein said preparing the substrate in step 2comprises: placing a polymer solution 2 into a mold, performing photo-crosslinking on the polymer solution 2 and the array of microneedles obtained in step 1, and demolding, to obtain the substrate-microneedle array structure,
wherein a polymer in the polymer solution 2 comprises at least one selected from a polymer with a photo-crosslinking group, a polymer monomer molecule with a photo-crosslinking group, and a composite of a polymer without a photo-crosslinking group and a polymer monomer molecule with a photo-crosslinking group, wherein:
the polymer with a photo-crosslinking group comprises at least one selected from methacrylated gelatin, methacrylated chitosan, polyether F127 acrylate, or polyethylene glycol acrylate;
the polymer without a photo-crosslinking group comprises at least one selected from polyvinyl alcohol, sodium alginate, chitosan, or cellulose; and
the polymer monomer molecule with a photo-crosslinking group comprises at least one selected from acrylamide, acrylic acid, N,N-methylene acrylamide, or methyl acrylate.

15. The method according to claim 12, wherein step 3 further comprises:
preparing a natural water-absorbing polymeric resin, placing the natural water-absorbing polymeric resin inside the substrate of the substrate-microneedle array structure prepared in step 2 to form the closed sandwich structure, to obtain the microneedle drug delivery apparatus,
wherein in step 3, the natural water-absorbing polymeric resin is placed in a cavity formed by the substrate, and the natural water-absorbing polymeric resin is connected to the substrate using biologic glue or by means of hot pressing,
wherein a raw material for preparing the liquid-absorbing swellable material in step 3 comprises a water-absorbing polymer and a crosslinking agent, wherein:
the water-absorbing polymer comprises at least one selected from sodium carboxymethyl cellulose, carboxymethyl chitosan, sodium carboxymethyl alginate, or hyaluronic acid; and
the crosslinking agent comprises at least one selected from citric acid, 1-ethyl-(3-dimethylaminopropyl)carbodiimide, glutaraldehyde, or genipin.

16. The method according to claim 13, wherein said photo-crosslinking in step 1 comprises:
mixing the drug, the polymer solution 1, and a photoinitiator 1 to obtain a photo-crosslinking solution;
dropwise adding the photo-crosslinking solution into the needle tip position of the mold;
filling the photo-crosslinking solution at the needle tip position sufficiently, by means of ultrasound, centrifugation, or evacuation; and
performing crosslinking, wherein the photoinitiator 1 comprises at least one selected from benzoin and derivatives thereof, benzoyl compounds, alkyl phenones, acylphosphine oxides, or benzophenones.

17. The method according to claim 14, wherein said photo-crosslinking in step 2 comprises:
mixing the polymer solution 2 with a photoinitiator 2;
placing the mixture in a mold; and
performing ultraviolet crosslinking in an ice-water bath, wherein the photoinitiator 2 comprises at least one selected from benzoin and derivatives thereof, benzoyl compounds, alkyl phenones, acylphosphine oxides, or benzophenones.

18. The method according to claim 14, wherein step 2 further comprises, subsequent to said photo-crosslinking:
performing a freeze-thaw cycling treatment.

19. A medicament, comprising:
the microneedle drug delivery apparatus according to claim 1; and
an active pharmaceutical ingredient.

20. A method for preparing a medicament, the method comprising:
placing an active pharmaceutical ingredient into microneedles of the microneedle drug delivery apparatus according to claim 1.
